(19) United States
(12) Patent Application Publication
(10) Pub. No.: US 2022/0241560 A1
(43) Pub. Date: Aug. 4, 2022

(54) CATHETERS AND INTERLOCKING RESTRAINT SYSTEMS THEREFOR

(71) Applicant: Securisyn Medical, LLC, Littleton, CO (US)

(72) Inventors: ARTHUR KANOWITZ, Littleton, CO (US); MARK BRUNING, Monument, CO (US); GREG LETENDRE, USAFA, CO (US)

(73) Assignee: Securisyn Medical, LLC, Littleton, CO (US)

(21) Appl. No.: 17/615,584

(22) PCT Filed: Jun. 10, 2020
(86) PCT No.: PCT/US2020/037083

§ 371 Date: Nov. 30, 2021

Related U.S. Application Data
(60) Provisional application No. 62/859,569, filed on Jun. 10, 2019.

(51) Int. Cl. A61M 25/02; A61M 16/04; A61M 1/04; A61D 7/00

(52) U.S. Cl. CPC A61M 25/02; A61M 16/0497; A61M 16/0465; A61M 2025/0266; A61D 7/00; A61M 2025/0253; A61M 1/04

(57) ABSTRACT

An interactive restraint system for securing catheters in position following installation in a human or animal patient and preventing movement or unplanned removal thereof in response to the application of significant forces in any direction thereto, be they longitudinal, torsional/rotational or bending. The system includes a retention collar which can be secured to any catheter and releasably interacts with a securing device attached to a patient to ensure proper positioning of the catheter. The restraint system includes a unique universal, after-market applied retention collar readily adapted for use in hospital and field emergency situations to engage any catheter and facilitate ease of application and positioning under all conditions.

### CROSS-REFERENCE TO RELATED APPLICATIONS

This application claims priority to U.S. Provisional Patent Application No. 62/859,569 filed on Jun. 10, 2019, the entire disclosure of which is incorporated herein by reference.

### FIELD OF THE INVENTION

The present invention relates generally to human and veterinary medical devices. Specifically, the present invention relates to catheters and tubular medical devices for insertion into the body of a human patient or, in veterinary applications, into the body of an animal and to an interlocking restraint system adapted to maintain a catheter in position. More specifically, the present invention relates to catheters (Intravenous, Foley), cricothyrotomy tubes, chest tubes, endotracheal tubes (“ETT's”), gastrostomy tubes, peritoneal catheters and other tubular medical devices and an interlocking or interactive restraint system adapted to maintain aftermarket tubular medical devices in position in the trachea, pleural space, mediastinum, pericardium, peritoneum, bladder or other spaces, body cavities, passageways, arteries or veins of a human patient or an animal for drainage or for the delivery of oxygen, medications or fluid infusions. The interlocking or interactive restraint system further prevents unintentional movement and/or removal of a tubular medical device from a human patient or animal, particularly in emergency and/or field situations or in any situation where an unintentional force may be applied to remove the tube.

### BACKGROUND OF THE INVENTION

Catheters are used to drain fluids from the body; for example, chest tubes for draining air or fluid from the area between a patient's chest wall and his or her lungs, Foley catheters which are used to drain the bladder, or peritoneal catheters which are inserted into the abdominal or peritoneal cavity during a dialysis procedure to circulate a cleansing fluid and to withdraw waste products therefrom. In contrast thereto, catheters are also used to infuse fluids or medications into the body; for example, intravenous (IV) catheters for infusing fluids, medications, or blood; gastrostomy tubes for infusing feeding into the stomach and endotracheal, tracheostomy, and cricothyrotomy tubes which are tubular medical devices designed to deliver ventilatory air/oxygen (as opposed to draining fluids, medications) to a human or animal patient.

Endotracheal intubation, tracheotomy, and cricothyrotomy are medical procedures used to place an airway device (artificial airway) into a patient's trachea or airway. The use of an airway device is mandated in situations where an individual, or an animal in veterinary applications, is unable to independently sustain the natural breathing function or maintain an open airway due to unconsciousness, trauma, disease, drugs or anesthesia. Thus, life-saving mechanical ventilation is provided through the airway device, which may be in the form of an endotracheal tube (EU), a tracheostomy tube or a cricothyrotomy tube, or several other commercially available supraglottic airway devices, depending upon the condition of the patient and the specific situation requirements.

Endotracheal intubation is accomplished by inserting an airway device into the mouth, down through the throat and larynx, and into the trachea. Tracheotomy and cricothyrotomy procedures involve making an incision in the trachea or in the cricothyrold membrane respectively into which a tube is inserted to create an airway to deliver ventilatory air in life-threatening or emergency situations where an airway is necessary but endotracheal intubation is impossible. These procedures create an artificial passageway through which air can freely and continuously flow in and out of a patients lungs and which prevents the patient's airway from collapsing or occluding.

Catheters by their very structure are passive devices, inasmuch as they do not include built-in features designed to secure them in proper position following insertion in a patient. However, it is critically important that any catheter be positioned accurately and maintained in the correct position in a patient. Were an airway device to move out of its proper position in the trachea and into the right main stem bronchial tube, only one lung will be ventilated. Failure to ventilate the other lung can lead to a host of severe pulmonary complications. Moreover, if the airway device moves completely out of the trachea and into the pharynx, esophagus or completely outside the body, the patient will become hypoxic due to the lack of ventilation to the lungs, a condition which typically results in life-threatening brain injury within a matter of only a few minutes.

Even after a catheter, for example, an airway device, has been positioned correctly, subsequent movement of the patient can lead to inadvertent movement of the device, as hereinabove described. An intubated patient may restlessly move about and may also attempt to forcibly remove an airway device, whether conscious or subconscious, particularly if the patient is uncomfortable or having difficulty breathing, which can lead to panic. In the case of an animal patient, agitation may be particularly pronounced due to the animal's lack of cognitive awareness or understanding of its circumstances and an instinctual survival fight or flight response. A large animal or a carnivore can pose a serious danger not only to itself but also to a treating veterinarian and anyone in close proximity under such circumstances.

Chest tubes are elongate, semi-flexible tubes or catheters that a treating physician or a surgeon may insert in the area between a patient's chest wall and his or her lungs, an area known as the pleural space. Chest tubes are used to address a number of emergency and post-operative conditions such as a collapsed lung, a buildup of excess blood or other fluid in the chest cavity, or to treat an infection, among others. Chest tubes have been widely recognized as life-saving devices in field medical scenarios where military personnel may experience a collapsed lung as the result receiving a chest wound.

Medical emergencies may occur anywhere. Accordingly, emergency medical service personal (i.e., paramedics) may be called upon to insert airway devices and/or chest tubes in out-of-hospital emergency settings, for example at accident scenes, military personnel in combat situations, emergency department physicians, anesthesiologists, and critical care clinicians in emergency response vehicles, as well as in hospital settings. Unintentional movement of an airway device, a chest tube or a catheter of any type is not uncommon, particularly when the patient is moved from an out-of-hospital or field setting to an emergency department of a hospital. Further, anytime a patient is moved, for example, not only from an ambulance to a trauma facility, but also from one hospital to another hospital, from one area of the hospital to another area in the same hospital (imaging, laboratory, operating theater), or from a hospital to an outpatient rehabilitation facility, unintentional movement of a catheter is a risk. Even repositioning a patient in a hospital bed, or in the case of an animal, in a recovery cage, may cause unintentional movement of any or all of the afore-mentioned devices.

U.S. Pat. No. 8,001,969 issued on Aug. 23, 2011, and U.S. Pat. No. 8,739,795 issued on Jun. 3, 2014, both to Arthur Kanowitz, the inventor of the present invention, disclose interlocking restraint or airway stabilization systems which address many of the problems set forth above associated with unplanned extubation of a patient intubated with an endotracheal tube (ETT). Continuing research into ways of providing even more advanced and rapidly deployable interlocking restraint or airway stabilization systems have resulted in yet further improvements to the overall design of ETT system components. Significantly, this work has led to an expanded investigation of interlocking restraint systems adapted to cooperatively interact with catheters in diverse applications to maintain the devices' correct positions in a patient, and to prevent unintentional movement and/or removal thereof from a patient or animal, particularly in emergency and/or field situations.

In view of the above, it will be apparent to those skilled in the art from this disclosure that a need exists for improved catheters and interactive restraint systems therefor for human and animal catheter treatment systems adapted for field, emergency and post-procedure situations that enable treating physicians, veterinarians and emergency medical response personnel to deliver life-saving and sustaining ventilatory air, medications, plasma, or, in a situation requiring the insertion of a chest tube, to treat an impaired respiratory function by draining fluid or air from a patient's pleural space, under diverse and potentially unpredictable situations and circumstances. The improved systems not only advantageously protect a catheter from occlusion and crushing, but also maintain the tubular devices in its respective preferred position in a patient or animal and prevents clinically significant movement thereof as a result of the application of multidirectional forces of significant magnitude thereto. The present invention addresses these needs in the art as well as other needs, all of which will become apparent to those skilled in the art from the accompanying disclosure.

### SUMMARY OF THE INVENTION

In order to address the aforementioned needs in the art, catheters and interactive restraint systems therefor are provided which may be used to treat human (or animal patients in veterinary applications) to drain and/or to deliver fluids, to maintain an airway or, in the situation requiring the insertion of a chest tube, to facilitate its insertion into and to maintain its position in the anatomical area selected by the treating physician or emergency responder. The individual interactive restraint systems disclosed herein prevent clinically significant movement of passive catheters such as cricothyrotomy tubes, tracheostomy tubes, chest tubes, and the like in response to the application of forces in any direction thereto, namely, longitudinal, torsional/rotational or bending.

Unlike conventional prior art aftermarket devices, the interactive restraint systems of the present invention comprise interactive components adapted to secure a catheter in position on a patient and restrain it against movement which may arise as a result of unintentionally applied forces thereto. Exemplary catheters include those designed for delivering air, fluids or medications, such as peripheral or central venous IV catheters, tracheostomy tubes, cricothyrotomy tubes and the like and other catheters for draining fluids from human or animal patients, by way of example, Foley catheters, peritoneal catheters, nasogastric tubes or chest tubes. Such catheters are in and of themselves stand-alone, passive devices that are susceptible to being displaced when bumped or otherwise exposed to unintentionally applied external forces. However, when such catheters are used in connection with the interlocking restraint system of the present invention the aforementioned problems associated with the prior art systems are virtually eliminated via the active interlocking stabilization components that cooperate integrally with and engage one another to provide unparalleled strength and stability against movement, even when the smooth surfaces of the catheter tubes become slippery from fluids and/or secretions, without applying any constricting pressure whatsoever to the tubular elements.

In an embodiment, a universal interlocking restraint system interacts with a retention member or collar by urging it into securing engagement with any smooth catheter tube via several methods that adhere, bond, or otherwise attach the collar to the tube. Following positioning adjustment, the collar is attached to the patient via an encapsulating tower and an apparatus such as a strap, adhesive pad, and the like adapted to secure the system to a patient. The collar and tower interact to allow adjustability while preventing movement of the collar relative to the tower.

In another embodiment, a cricothyrotomy tube has a semi-rigid elongate body which conforms to a patient's trachea after it is inserted into the patient and includes a continuous sidewall extending between a machine end and a patient end portion thereof, thereby forming a hollow conduit through which the airway is established. A retention member or collar is positioned on the exterior of the sidewall of the device between the end portions thereof at a predetermined fixed position relative to the patient end of the airway device and adjacent the external incision made in a patient's skin and cricothyroid membrane through which the tube is inserted. The retention collar includes one or more spaced-apart alternating ribs and structural recesses extending circumferentially about the body of the collar. At least one rib and structural recess provide an active surface area forming a tight interlocking fit with cooperating interlocking flanges and structural recesses of an interlocking restraint system secured to the patient, thereby establishing a complete barrier against movement of the device to which it is connected resulting from forces applied to the device as hereinabove described.

In still another embodiment, a retention member or collar includes an inner diameter or circumference which is sized to fit the outer diameter or circumference of the catheter on which it will be positioned.

In another embodiment, a chest tube is similarly constructed, having a flexible elongate body which is slightly curved for insertion through an incision made in a patient's chest and into the patient's pleural or intrathoracic space and includes a continuous sidewall extending between a machine end and a patient end portion thereof, thereby forming a hollow conduit through which air, blood, and other fluids are drawn out of the space. The patient end has an aperture formed therein and a series of at least two apertures formed in the portion of the continuous sidewall extending longitudinally from the patient end for the air, blood, and fluids to enter the tube. Distance markers are positioned along the length of the tube indicating the distance from the last aperture in the patient end.

In still another embodiment, an interlocking restraint system includes a securing apparatus or stabilizer having a base plate or ring, a tower structure operatively connected thereto, and, in a chest tube system, an adhesive pad attached to the base plate and which extends partially circumferentially around the tower structure in a plane perpendicular to the longitudinal axis of the tower structure. The tower structure is configured to cooperate with the interacting retention collar on a catheter to prevent clinically significant movement of the catheter after it is inserted in a patient.

In another embodiment, a base plate is formed of a single ring or washer-shaped member adapted to fit over a tower structure and a cricothyrotomy tube with which it interacts to allow greater ease of application, the base plate or ring being structured and arranged to be secured on a patient's throat area adjacent an incision into which the cricothyrotomy tube is inserted or adjacent the incision in a patient's chest into which the chest tube is inserted. The systems may be retained in place by a securing device, for example, an adjustable strap which is attached thereto, and which extends around either a patient's neck or torso.

In yet another embodiment, the tower structure is secured to the base ring and extends distally outwardly therefrom in a direction away from a patient. The tower includes a pair of oppositely disposed pivotally interconnected c-shaped collars, each collar including at least one annular flange and structural recess positioned axially along the inner surface of the body portion of each of the collars extending substantially inwardly therefrom, the at least one rib and structural recess of the retention collar operatively interacting with the annular flange and structural recess of the restraining tower to retain a catheter in position on a patient via releasable engagement with the retention member secured thereto.

In an embodiment, a securing apparatus may be installed on and/or removed from an aftermarket catheter positioned previously in a patient without interrupting the function or operation of the catheter.

In yet another embodiment, a securing apparatus includes a retention member or collar having and inner surface adapted to be releasably engaged with a catheter, the inner surface being structured and arranged to securely grip a catheter, whereby clinically significant movement of the catheter with respect to a patient is prevented.

These and other features, aspects and advantages of the present invention will become apparent to those skilled in the art from the following detailed description of preferred embodiments taken in connection with the accompanying drawings, which are summarized briefly below.

### BRIEF DESCRIPTION OF THE DRAWINGS

Referring now to the attached drawings which form a part of this original disclosure:

FIG. 1 is a side plan view of the elements of a catheter in the form of a cricothyrotomy tube including portions of an interlocking restraint system in accordance with an embodiment of the present invention;

FIG. 2 is a partial sectional side view of a cricothyrotomy tube positioned in the trachea of a patient in accordance with an embodiment;

FIG. 3.A. is a top plan view of portions of an interlocking restraint system for a catheter shown in an open position to illustrate the elements thereof more clearly in accordance with an embodiment;

FIG. 3.B. is a side plan view of portions of the interlocking restraint system of FIG. 3.A.;

FIG. 4.A. is a top plan view of the interlocking restraint system of FIGS. 3.A. and 3.B. shown in a closed position;

FIG. 4.B. is a side plan view of portions of the interlocking restraint system of FIG. 4.A.;

FIG. 5 is a partial sectional side view side view of the cricothyrotomy tube of FIGS. 1 and 2 having an interlocking restraint system attached thereto in accordance with an embodiment;

FIG. 6 is a side plan view of the elements of a catheter in the form of a chest tube including portions of an interlocking restraint system according to an embodiment of the present invention;

FIG. 7.A. is a top plan view of portions of an interlocking restraint system for a chest tube catheter shown in an open position to illustrate the elements thereof more clearly in accordance with an embodiment;

FIG. 7.B. is a side plan view of the interlocking restraint system of FIG. 7.A.;

FIG. 8.A. is a top plan view of portions of the interlocking restraint system of FIGS. 7.A. and 7.B. shown in a closed position in accordance with an embodiment;

FIG. 8.B. is a side plan view of the interlocking restraint system of FIG. 8.A.;

FIG. 9 is a partial sectional side view of a chest tube positioned in a patient's chest cavity and having portions of an interlocking restraint system secured thereto in accordance with an embodiment;

FIG. 10 is a partial sectional side view of a chest tube positioned in a patient's chest cavity and having an interlocking restraint system secured thereto in accordance with an embodiment;

FIG. 11.A. is a top plan view of a retention member or collar portion of an interlocking restrain system shown in an open position in accordance with another embodiment;

FIG. 11.B. is a side plan view of the retention member or collar portion of FIG. 11.A.;

FIG. 12.A is a top plan view of a retention member or collar portion of FIGS. 11.A. and 11.B. shown in a closed position;

FIG. 12.B. is a side plan view of the retention member or collar portion of FIG. 12. A.;

FIG. 13.A. is still another top plan view of the retention member or collar portion shown in FIG. 12.A. rotated 90° to illustrate the elements thereof more clearly;

FIG. 13.B. is a side plan view of the retention member or collar portion of FIG. 13. A.;

FIG. 14.A. is a top plan view of the retention member or collar portion of an interlocking restrain system of FIG. 13.A. shown in an open position to illustrate the elements thereof more clearly in accordance with an embodiment;

FIG. 14.B. is a side plan view of the retention member or collar portion of FIG. 14.A.;

FIG. 15 is a perspective view of portions of an interlocking restraint system for an endotracheal tube shown in a closed position in accordance with an embodiment;

FIG. 16 is a perspective view of portions of the interlocking restraint system for an endotracheal tube of FIG. 15 shown in an open position to illustrate the elements thereof more clearly in accordance with an embodiment;

FIG. 17 is a perspective view of portions of the interlocking restraint system for an endotracheal tube of FIGS. 15 and 16 shown in an open position illustrating the removal of a protective layer covering an inner surface of a first semi-cylindrical retention collar thereof in accordance with an embodiment;

FIG. 18 is a perspective view of portions of the interlocking restraint system for an endotracheal tube of FIGS. 15-17 shown in an open position illustrating the removal of a protective layer covering an inner surface of a second semi-cylindrical retention collar thereof in accordance with an embodiment;

FIG. 19 is a perspective view of portions of the interlocking restraint system for an endotracheal tube of FIGS. 15-18 shown in an open position with the protective layers removed from the first and second semi-cylindrical retention collars in accordance with an embodiment;

FIG. 20 is a perspective view of portions of the interlocking restraint system for an endotracheal tube of FIGS. 15-19 shown in an open position with the endotracheal tube positioned therein in accordance with an embodiment;

FIG. 21 is a perspective view of portions of the interlocking restraint system for an endotracheal tube of FIGS. 15-20 shown in a partially-closed position on an endotracheal tube in accordance with an embodiment; and

FIG. 22 is a perspective view of portions of the interlocking restraint system for an endotracheal tube of FIGS. 15-21 shown in an open position with the first and second semi-cylindrical retention collars secured to an endotracheal tube in accordance with an embodiment.

### DETAILED DESCRIPTION OF THE PREFERRED EMBODIMENTS

Selected embodiments of the present invention will now be explained with reference to the drawings. It will be apparent to those skilled in the art from this disclosure that the following descriptions of the embodiments of the present invention are provided for illustration only and not for the purpose of limiting the invention as defined by the appended claims and their equivalents.

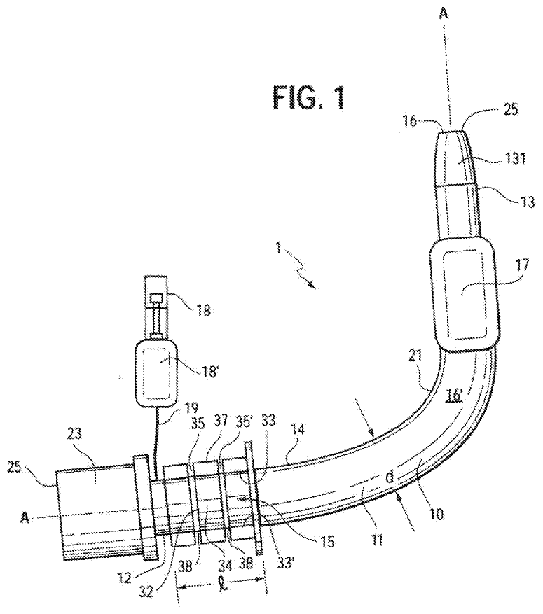

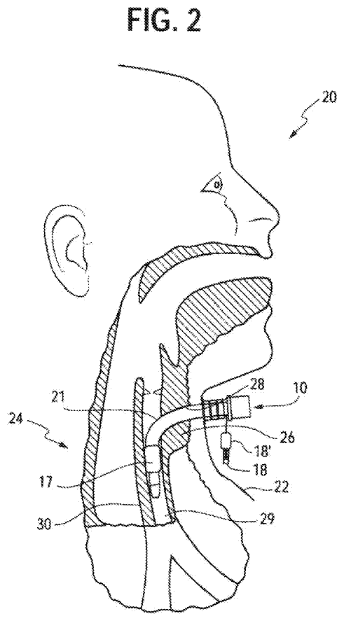

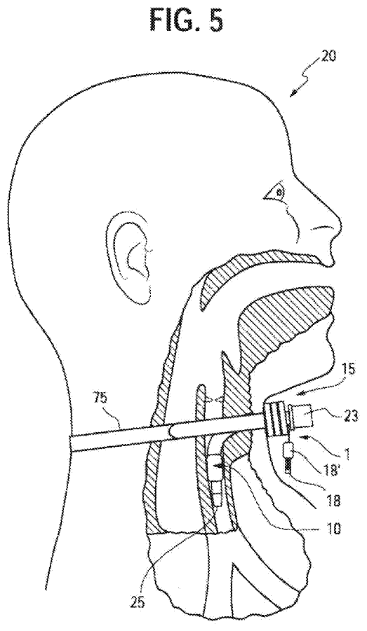

Referring initially to FIGS. 1 and 2, a catheter in the form of a cricothyrotomy tube 10 including a retention member or collar 15 of an interlocking restraint system 1 (shown installed on a human patient 20 in FIG. 5) is illustrated in accordance with an embodiment of the present invention. A cricothyrotomy tube is used to establish an airway in the patient 20 (or animal patient in veterinary applications) under conditions where natural respiration is impossible or severely compromised due to a severe illness or injury that prevents the patient from ventilating on his or her own and when a non-surgical airway is not successful. As shown in FIG. 2, a cricothyrotomy procedure involves making an incision through the skin 22 of the patient's neck (shown generally at 24) overlying the cricothyroid membrane 26. A second incision is made in the cricothyroid membrane thereby creating an aperture 28 in and access to an interior airway passage 29 formed by the patient's trachea 30. The cricothyrotomy tube is inserted through the skin incision and the aperture 28 into the airway passage 29 to provide an artificial airway for ventilatory support to the patient.

Referring again to FIG. 1, the cricothyrotomy tube 10 includes a semi-rigid, elongate cylindrical body 11 having an external diameter d and extending along and circumferentially about an axis A-A and having a machine end portion 12, a patient end portion 13 having a tapered tip 131 adapted to facilitate insertion into a patient and a continuous sidewall 14 having an internal surface 16 and an external surface 16′ extending intermediate the machine and the patient ends. An inflation balloon or cuff 17 is operatively connected to the patient end of the cylindrical body of the tube and adapted to be inflated following insertion into the patient's trachea by an inflation mechanism, for example, a pump or a syringe (not shown) connected thereto via an air flow control valve 18, pilot balloon 18′ that indicates the inflation pressure in the cuff 17, an inflation tube 19 and an inflation lumen (not shown) which may be formed in the continuous sidewall of the cylindrical body. The cricothyrotomy tube body has a bend 21 formed therein at a preselected location depending upon the size of the tube to facilitate insertion into and conformance with the anatomical shape of a patient's trachea, which is best shown in FIG. 2. A standard 15 mm connector 23 is operatively connected to the machine end portion 12 positioned outside the patient's body, the standard connector being structured and arranged to connect the cricothyrotomy tube to a source of ventilatory air. The ventilatory air is directed via a passage 25 formed by the cylindrical body's sidewall and extending the length of the tube intermediate the machine and patient ends 12 and 13. Cricothyrotomy tubes are provided in various sizes, the diameter d, circumference and length of a tube being determined by the anatomy of the patient being treated, which may range from small in size in the case of a pediatric patient or considerably larger in size to treating a mature adult.

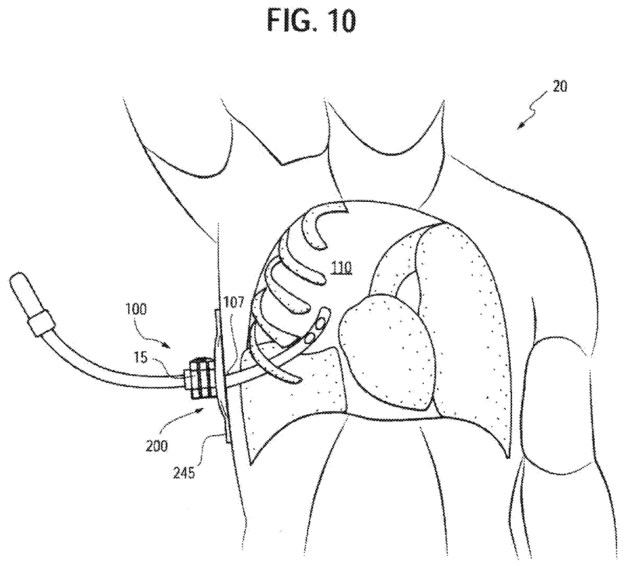

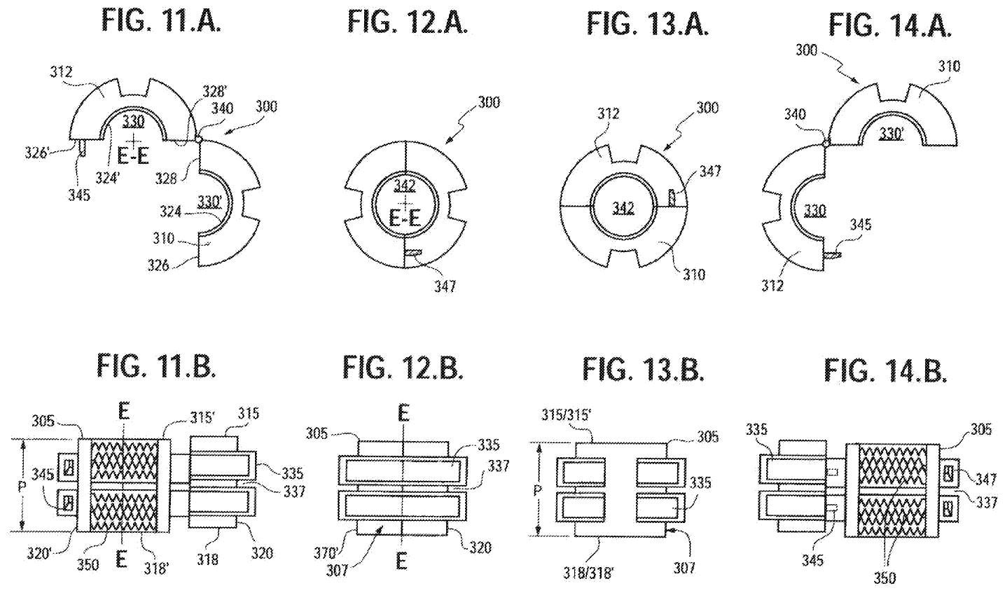

The retention member or collar 15 includes a cylindrically shaped body 32 having an aperture 33 extending therethrough adjustably positioned on the machine end 12 of the cricothyrotomy tube 10, the cylindrical shaped body of the collar extending circumferentially about and coaxially along the cricothyrotomy tube's cylindrical body 11. Certain catheter products such as the SolidAlRity™ Airway Stabilization System include an endotracheal tube catheter having a collar secured thereto by being permanently bonded during manufacture to the tube's cylindrical body, However, in accordance with the present invention, the collar is preferably applied to any catheter tube after positioning the tube in a patient, by any suitable means (bonding, PSA, gripping) to prevent relative movement between the collar and the tube, thus providing a universally-available, aftermarket retention collar. By way of example, the collar may be formed of a single piece of material or, alternatively, it may be formed or press fitted over the tube body, chemically bonded to the tube body or have an inner surface 33′ having various treatments adapted to operatively connect the retention member to the catheter. By way of example and not of limitation, the components may be adhesively connected using suitable adhesives or glues such as pressure sensitive adhesive (PSA), via double sided tape applied to an inner surface or via textured or scalloped surface patterns 350 formed thereon, the patterns being similar to those on the external surface of a porcupine quill, as shown in FIGS. 11.B. and 14.B. The retention member has a length “I” and includes at least one rib 34 having oppositely disposed sides 35, 35′ extending circumferentially around the body 32 of the collar 15 and extending radially outwardly therefrom and a flat end portion 37 operatively connected to and extending intermediate the sides 35, 35′ in a direction generally parallel to the axis A-A of the tube. The retention member further includes a pair of circumferentially extending structural recesses 38 positioned axially along the length of the retention member, each of the structural recesses being positioned adjacent to a respective side 35, 35′ of the at least one rib 34. In the embodiment shown, by way of example and not of limitation, the collar includes three ribs 34; however, it is to be understood that a collar may include fewer or more ribs and corresponding structural recesses, depending upon the application.

Cricothyrotomy tubes are provided in assorted sizes, the diameter d, circumference, and length of a tube being determined by the anatomy of the patient being treated. Accordingly, in an embodiment, a retention member or collar may be selectively provided with an inner diameter or circumference which is sized to fit over and in securing engagement with the outer diameter or circumference of the catheter on which it will be positioned. In this embodiment, the outer diameter or circumference of the collar remains unchanged, thereby facilitating application of a securing apparatus in any situation without the need for matching a particular securing apparatus with catheters of different external dimensions.

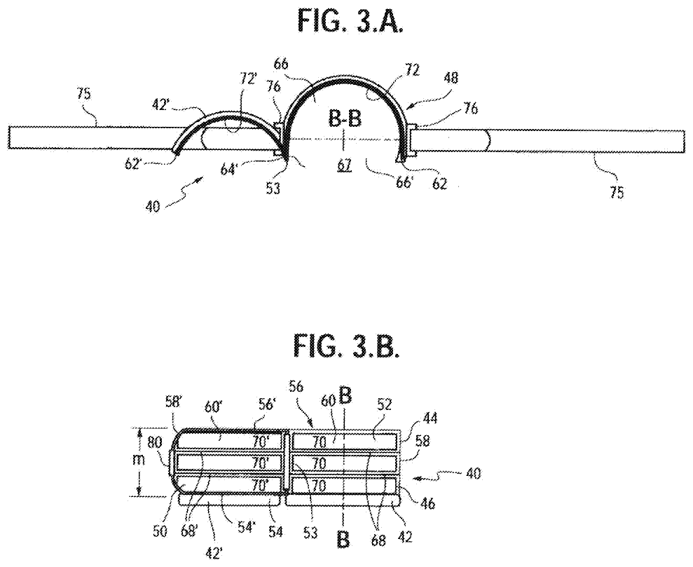

Referring to FIGS. 3.A. and 3.B, a securing apparatus portion of the interlocking restraint system is shown generally at 40. The securing apparatus is adapted to releasably engage the retention member or collar 15 and to cooperate therewith to maintain the cricothyrotomy tube in position in a patient's airway and to prevent movement or unplanned extubation of the patient as a result of multidirectional forces being applied to the tube.

The securing apparatus 40 includes a base plate or ring members 42, 42′ and a generally cylindrically shaped tower structure 44 having a longitudinal axis B-B hinged semi-circular sections or c-shaped collars 50, 52 operatively connected to the ring members 42, 42′ and extending in a substantially perpendicular direction from a top surface 46 thereof along axis B-B, as will be described in greater detail below. The tower structure 44 includes a body portion 48 having a length and including the oppositely disposed, pivotally interconnected, c-shaped collars 50, 52 respectively extending generally symmetrically about and along the axis B-B in a direction away from the patient's airway when installed on a patient. Each of the collars has a length m, first and second end portions 54, 54′ and 56, 56′, an outer surface 58, 58′, and an inner surface 60, 60′. Each of the outer and inner surfaces extends intermediate the c-shaped collars' respective first and second end portions. Each of the collars has a pair of generally parallel extending edge surfaces 62, 62′ and 64, 64′, the edge surfaces and the corresponding c-shaped collar each defining an opposed, semi-cylindrically shaped cavity 66, 66′ about the axis B-B. These cavities are most clearly shown in FIGS. 3.A. and 4.A. as seen from the top. The ring members 42, 42′ are secured to the first and second end portions 54, 54′ respectively of collars 50, 52.

Each c-shaped collar includes a plurality of substantially uniformly spaced-apart annular flanges 68, 68′ positioned axially along the respective inner surfaces thereof and extending substantially inwardly therefrom, and a plurality of structural recesses 70, 70′ positioned axially along each inner surface intermediate an adjacent two of the plurality of substantially uniformly spaced-apart annular flanges, each one of the plurality of annular flanges cooperating with an adjacent one of the plurality of annular flanges to define one of the plurality of structural recesses. Each of the annular flanges has an aperture 72, 72′ formed therein, each aperture being adapted to receive the cricothyrotomy tube and a retention member when installed thereon, as will be described in greater detail below. The securing apparatus may be secured to the patient's throat area by a suitable attachment apparatus, by way of example and not of limitation, a strap 75 secured to the base plate or ring 42 at attachment apertures or loops 76 and further extending around the patient's head and securable by buckles, Velcro or other suitable attachments, as is known in the art.

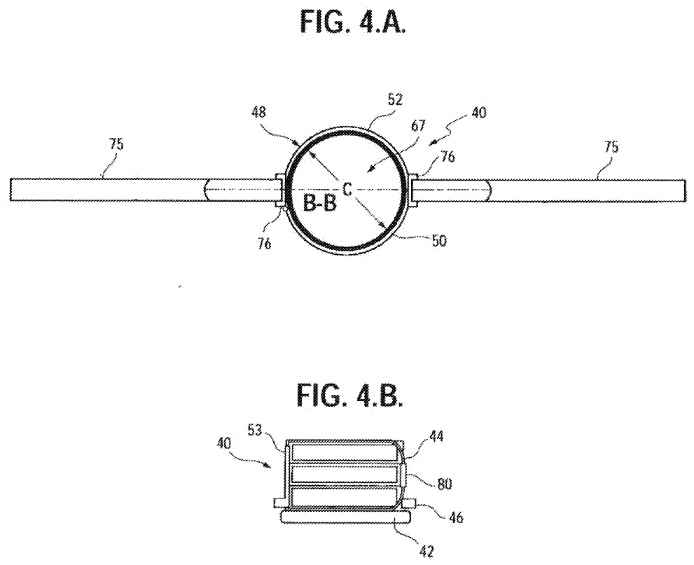

In operation. the collars 50 and 52 are pivotally interconnected, for example, by hinge member 53 and are moveable into mating contact with one another, thereby forming a cavity 67 defined by the opposed semi-cylindrically shaped cavities 66, 66′, the cavity having an inner diameter C and being adapted to releasably engage and enclose the retention member secured to a catheter, which in the instant embodiment comprises a cricothyrotomy tube, such that axis A-A of the tube extends coaxially along axis B-B of the cylindrically shaped tower structure 44. Each of the c-shaped collars includes a snap, clip, latch, camming operating apparatus or other suitable interlocking feature 80 having one or more locking members adapted to releasably engage corresponding mating locking members formed in or secured to the other c-shaped collar to releasably clamp them together circumferentially around the tube and collar in stabilizing and supporting engagement therewith. A release mechanism, for example, a quick-release actuator or button as is known in the art, allows the c-collars to be easily and rapidly released from locking engagement with one another to facilitate positioning and adjustment of the components of the interlocking restraint system. Once the cricothyrotomy tube is inserted into and positioned in a patient's trachea, the strap 75 of the securing apparatus is secured around the patient's neck. When the c-collars 50 and 52 are locked together as shown in FIG. 4.B., the inwardly extending annular flanges 68, 68′ and structural recesses 70, 70′ on the inner surfaces of the of the c-shaped collars releasably engage the at least one outwardly extending rib 34 corresponding mating structural recesses 38 of the retention member, thereby creating multiple points of contact and interaction between the securing apparatus and the catheter, thus preventing clinically significant movement of the thereof in response to substantial forces which may be applied thereto in any direction. The retention structure and securing apparatus cooperate to completely enclose the tube whereby it is isolated totally from any constricting, pinching, or crushing forces that would constrict an inner diameter thereof, thereby also restricting ventilation of a patient. Advantageously, in accordance with an embodiment of the instant invention, unlike prior art securing devices, the system herein disclosed may be secured laterally from a side of a catheter without being placed over the end of the tubular portion thereof and, therefore, without interrupting a particular catheter's function.

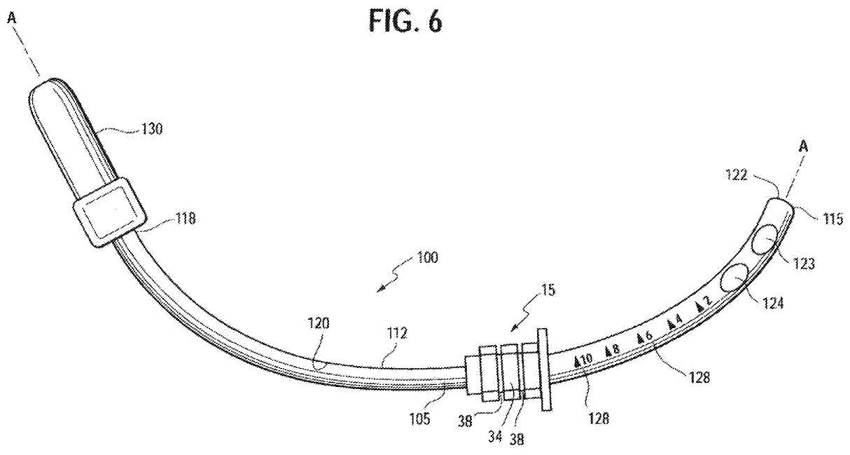

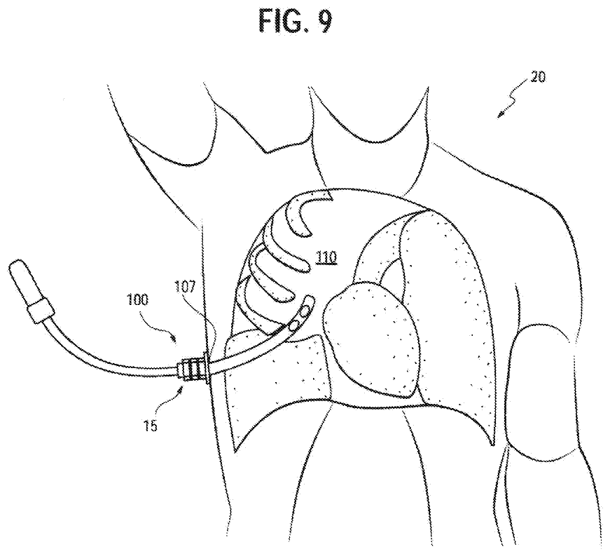

Turning now to FIGS. 6-10, the details of an embodiment of an interactive restraint system of the present invention used in connection with a catheter in the form of a chest tube are shown. FIG. 6 illustrates the specific elements of a chest tube 100. The chest tube includes a flexible elongate body 105 which is slightly curved for insertion through an incision 107 made in a patient's chest and into the pleural or intrathoracic space 110 of patient 20, as best shown installed in a patient in FIGS. 9 and 10. The elongate cylindrical body includes a continuous sidewall 112 extending between a patient end 115 and a machine end 118 thereof, thereby forming a hollow conduit 120 through which air, blood, and other fluids are drawn out of the space 110. The patient end has an aperture formed therein and a series of at least two apertures 123 and 124 formed in the portion of the continuous sidewall extending longitudinally from the patient end for the air, blood, and fluids to enter the tube. Distance markers 128 are positioned along the length of the tube indicating the distance from the aperture located the furthest from the patient end to the surface of the patient's chest. A cap 130 is operatively connected to the machine end of the chest tube to prevent blood from squirting out of the end of the tube once it is placed in a patient's chest. After the chest tube is stabilized, the cap is removed, and the chest tube is connected to a pump or other suitable apparatus for removing fluids from the body cavity 110.

The retention member or collar 15 as described above with respect to the cricothyrotomy tube embodiment of FIGS. 1 and 2 may also be used in the chest tube embodiment of FIG. 6. The collar 15 is positioned on and extends circumferentially about and coaxially along the chest tube's elongate body 105. The collar may be secured to the tube's body by any suitable means to prevent relative movement therebetween after installation of the tube in a patient, the specifics of which are discussed above and incorporated herein by reference.

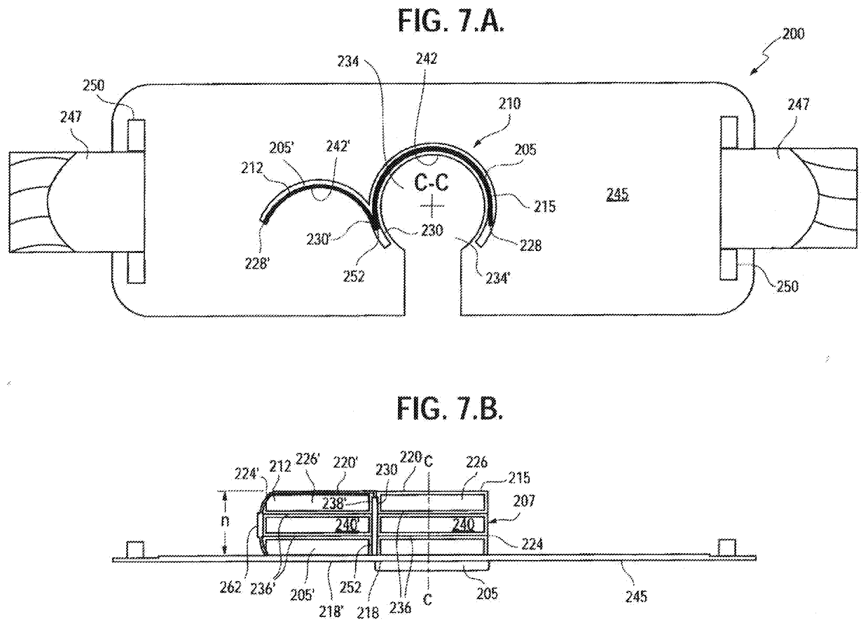

Referring now to FIGS. 7.A. and 7.B, a securing apparatus portion of the interlocking restraint system is shown generally at 200. The securing apparatus is adapted to releaseably engage the retention member or collar and to cooperate therewith to maintain the chest tube in position in a patient's pleural or intrathoracic space 110 and to prevent movement or unplanned extubation of the patient as a result of multidirectional forces being applied to the tube.

The securing apparatus 200 is similar in structure and configuration to the securing apparatus 40 discussed above and includes base plate or ring members 205, 205′ and a generally cylindrically shaped tower structure 207 operatively connected to the ring members and extending in a substantially perpendicular direction from a top surface thereof along axis C-C. The tower structure includes a body portion 210 having a length and including a pair of oppositely disposed, pivotally interconnected, c-shaped collars 212, 215 respectively extending generally symmetrically about and along the axis C-C in a direction away from the patient's chest when installed on a patient. Each of the collars has a length n, first and second end portions 218, 218′ and 220, 220′, an outer surface 224, 224′, and an inner surface 226, 226′. Each of the outer and inner surfaces extends intermediate the c-shaped collars' respective first and second end portions. Each of the collars has a pair of generally parallel extending edge surfaces 228, 228′ and 230, 230′, the edge surfaces and the corresponding c-shaped collar each defining an opposed, semi-cylindrically shaped cavity 234, 234′ about the axis C-C. These cavities are most clearly shown in FIG. 7.A. Ring member 205 is secured to first end portion 218 of collar 215, and ring member 205′ is secured to first end portion 218′ of collar 212.

Each c-shaped collar includes a plurality of substantially uniformly spaced-apart annular flanges 236, 236′ positioned axially along the respective inner surfaces thereof and extending substantially inwardly therefrom, and a plurality of structural recesses 240, 240′ positioned axially along each inner surface intermediate an adjacent two of the plurality of substantially uniformly spaced-apart annular flanges, each one of the plurality of annular flanges cooperating with an adjacent one of the plurality of annular flanges to define one of the plurality of structural recesses. Each of the annular flanges has an aperture 242, 242′ formed therein, each aperture being adapted to receive the chest tube and a retention member when installed thereon. The securing apparatus may be secured to the patient's chest area by a suitable attachment apparatus, by way of example and not of limitation, an adhesive pad 245. Optionally, a strap 247 may be secured to the adhesive pad at attachment apertures 250, the strap being adapted to extend around the patient's torso and to be secured by buckles, Velcro® or other suitable attachments, as is known in the art.

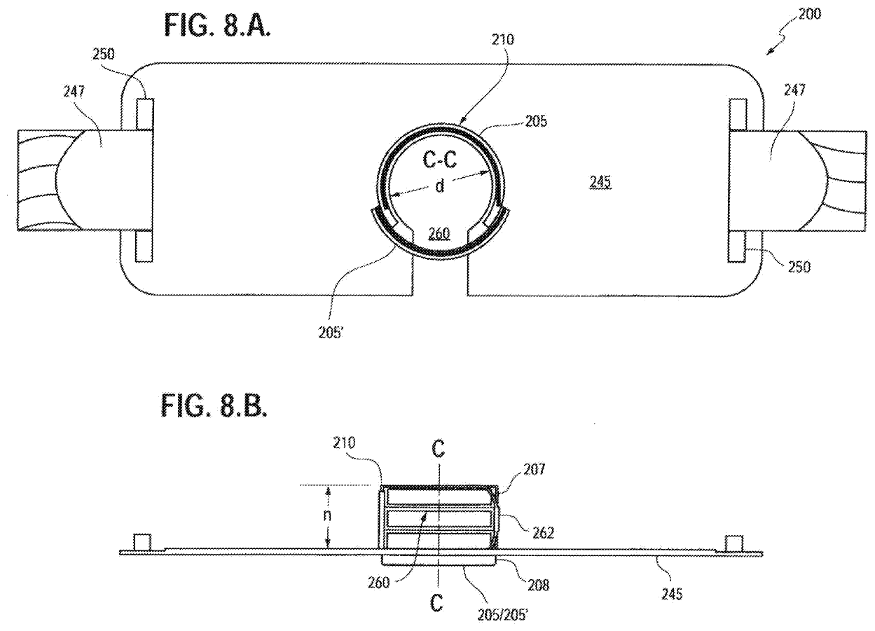

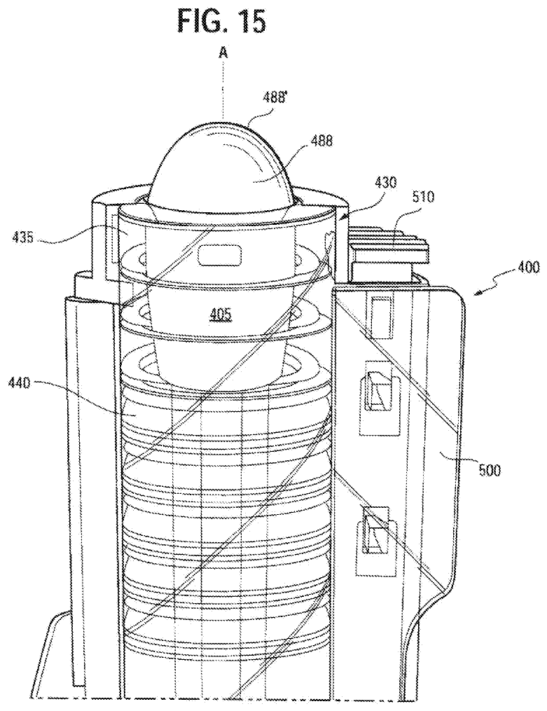

In operation. the collars 212 and 215 are pivotally interconnected, for example, by hinge member 252 and are moveable into mating contact with one another, thereby forming a cavity 260 defined by the opposed semi-cylindrically shaped cavities 234, 234′, the cavity having an inner diameter D and being adapted to releasably engage and enclose the retention member or collar secured to a chest tube, such that axis A-A of the tube extends coaxially along axis C-C of the cylindrically shaped tower structure, as best illustrated in FIG. 9. Each of the c-shaped collars includes a snap, clip, latch, ramming operating apparatus or other suitable interlocking feature 262 having one or more locking members adapted to releasably engage corresponding mating locking members formed in or secured to the other c-shaped collar to releasably clamp them together circumferentially around the tube and collar in stabilizing and supporting engagement therewith. A release mechanism, for example, a quick-release actuator or button as shown in the embodiment of FIGS. 15-22, allows the c-collars to be easily and rapidly released from locking engagement with one another to facilitate positioning and adjustment of the components of the interlocking restraint system. When the c-collars 50 and 52 are locked together as depicted in FIGS. 8.A. and 8.B., the inwardly extending annular flanges 236, 236′ and structural recesses 240, 240′ on the inner surfaces of the of the c-shaped collars releasably engage the at least one outwardly extending rib 34 corresponding mating structural recesses 38 of the retention member, thereby creating multiple points of contact and interaction between the securing apparatus and the catheter, thus preventing clinically significant movement of the thereof in response to substantial forces which may be applied thereto in any direction. The retention structure and securing apparatus cooperate to completely enclose the tube whereby it is isolated totally from any constricting, pinching, or crushing forces that would constrict an inner diameter thereof, thereby also restricting drainage of fluids from the chest cavity.

In field emergency situations such as those that may be encountered by backcountry paramedics, ski patrol personnel, and military medics in combat situations, for example, the components interactive stabilization apparatus of the embodiments of FIG. 1, et seq. may not be readily available and may not be included as part of routinely carried field gear due to size and weight limitations. Accordingly, a relatively small, compact and lightweight catheter interactive stabilization device that could be carried into remote areas in a field emergency first aid pack, a fanny pack or even in a pocket of a field jacket or field pants could save an accident victim's life while awaiting evacuation to a trauma facility.

Referring to FIGS. 11-14, an embodiment of a retention collar 300 is illustrated which meets the foregoing needs and which may be used with exemplary catheters such as cricothyrotomy tubes, tracheostomy tubes and chest tubes shown in FIGS. 1 and 6. The collar includes a generally cylindrically-shaped tower structure 305 extending along axis E-E, the tower structure including a body portion 307 having a length p and comprising a pair of oppositely disposed, pivotally interconnected, c-shaped collars 310, 312 respectively extending generally symmetrically about and along the axis E-E in a direction away from a patient when installed on a catheter apparatus. Each of the collars has first and second end portions 315, 315′ and 318, 318′, an outer surface 320, 320′, an inner surface 324, 324′. Similar in construction to the tower of the embodiment of FIG. 1, each of the outer and inner surfaces extends intermediate the c-shaped collars' respective first and second end portions. Each of the collars has a pair of generally parallel extending edge surfaces 326, 328 and 326′, 328′, the edge surfaces and the corresponding c-shaped collar each defining a semi-cylindrically shaped cavity 330, 330′ about the axis E-E. These cavities are most clearly shown in FIGS. 11.A. and 14.A.

Each c-shaped collar includes at least one annular rib or flange 335 having oppositely disposed sides 335′ positioned axially along the respective outer surfaces thereof and extending circumferentially about substantially outwardly therefrom. In the embodiment shown, each collar includes two annular flanges and a structural recess 337 positioned axially along each outer surface intermediate the spaced-apart annular flanges.

In operation, the collars 310 and 312 are pivotally interconnected, for example, by hinge member 340 and are moveable into mating contact with one another, thereby forming a cavity 342 when closed, the cavity being adapted to releasably receive a catheter such as a chest tube and the like. Each of the c-shaped collars includes a snap, clip, latch, camming operating apparatus or other suitable interlocking feature 345 adapted to releasably engage corresponding mating locking members 347 formed in or secured to the other c-shaped collar to releasably clamp them together circumferentially around the catheter in stabilizing and supporting engagement therewith. A release mechanism, for example, a quick-release actuator or button as described above, or simply an interlocking snap device allows the c-collars to be easily and rapidly released from locking engagement with one another to facilitate positioning of collar on a catheter.

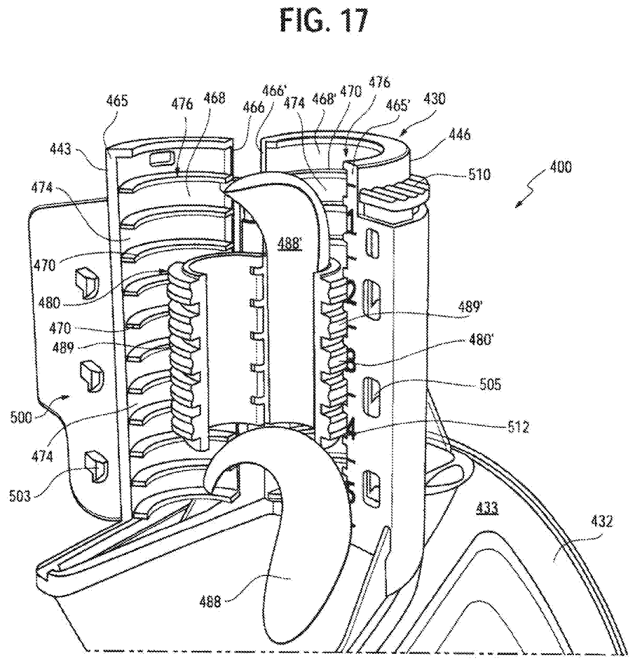

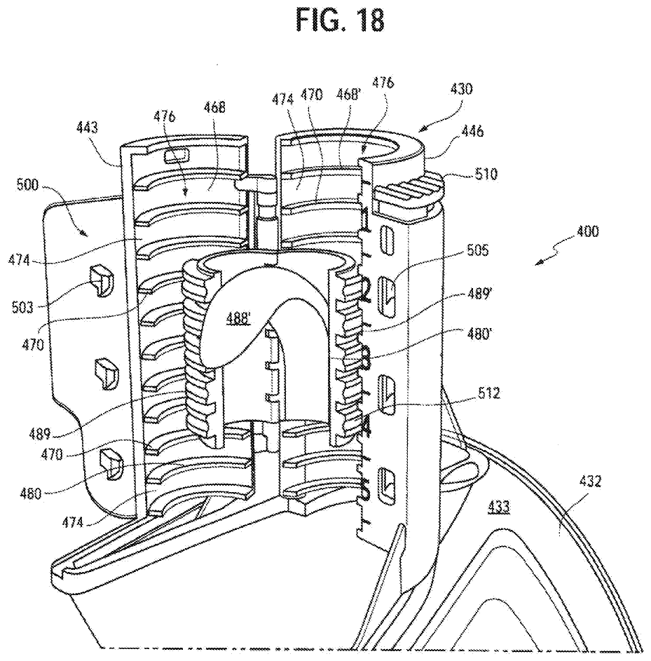

The collar may be secured to a catheter tube's cylindrical body by any suitable means to prevent relative movement therebetween after installation of the tube in a patient. By way of example, the collar may be adhesively connected during the installation process via a suitable bonding agent such as double sided tape or a pressure-sensitive adhesive applied to the inner surfaces 324, 324′ or via surface texturing such as scalloped surface patterns 350 formed thereon as shown in FIGS. 11.B. and 14.B., the patterns being similar to those on the external surface of a porcupine quill. If double sided tape is used, a protective film may be removed from the exposed side immediately prior to positioning the collar on a catheter tube, as illustrated in the embodiment of FIGS. 17 and 18. The quill design could be combined with an adhesive design for added strength.

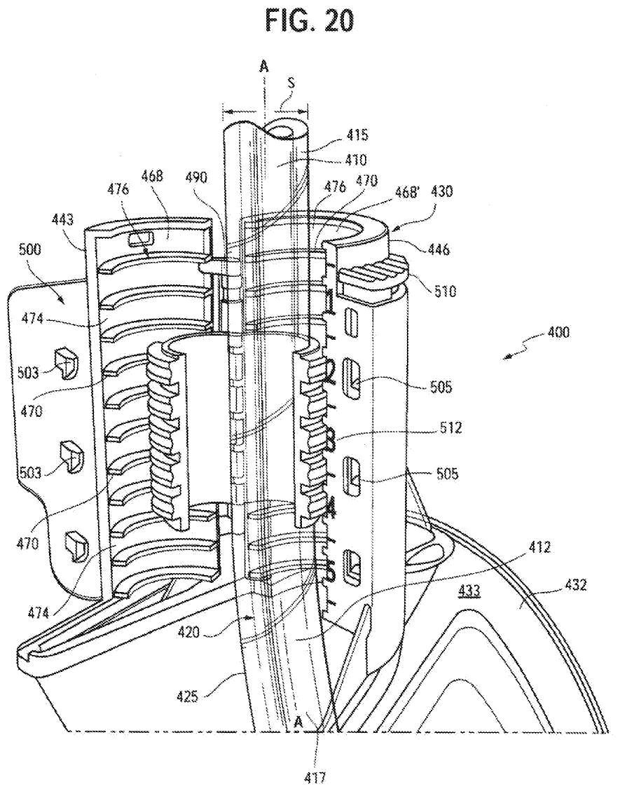

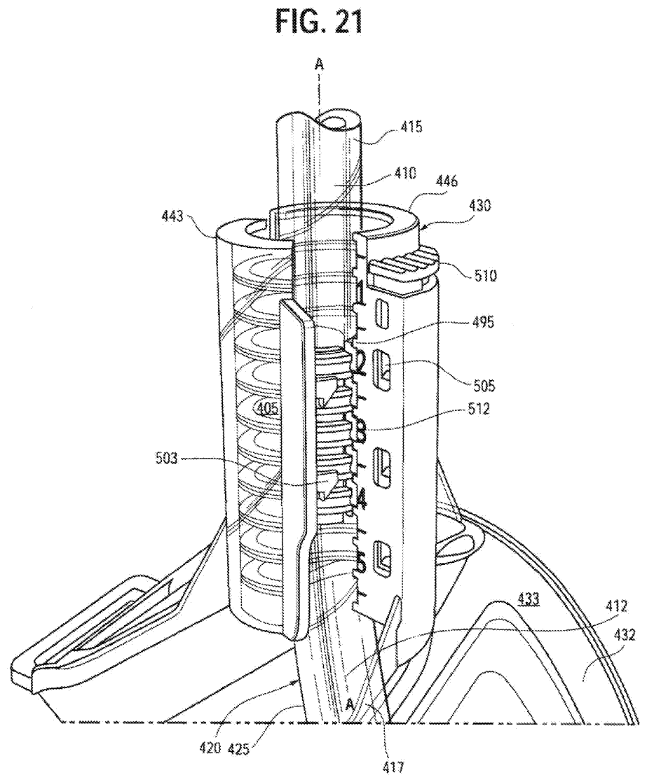

Referring now to FIGS. 15-22, an interlocking or interactive catheter restraint system shown generally at numeral 400 is illustrated in accordance with an embodiment of the present invention. In the instant embodiment, the restraint system is shown in the form of an airway stabilization system to stabilize an airway device 410 (FIG. 20) used to maintain an airway in a human (or animal patient in veterinary applications) under conditions where natural respiration is impossible or severely compromised. The restraint system includes an endotracheal tube 410 which has a flexible elongate body 412 extending along an axis A-A and having a length, an outer diameter S, a distal end portion 415, a proximal end portion 417 and a continuous sidewall 420 having an internal surface (not shown) and an external surface 425 extending between the proximal and the distal ends. Any one of several commercially available endotracheal tubes or any one of several commercially available supraglottic airway devices such as a King LT™ airway device manufactured by King Systems, Noblesville, Ind. or a laryngeal mask airway (LMA) such as a LMA Classic™ manufactured by LMA North America, San Diego, Calif. may be used without departing from the scope of the present invention.

Referring again to FIGS. 15-19, the interactive restraint system is depicted in greater detail and includes a securing apparatus 430 having a plate or faceplate 432 which may be secured to the patient's face by a suitable attachment apparatus, by way of example and not of limitation, a strap extending around the patient's head and securable by buckles, Velcro® or other suitable attachments, as is known in the art. The plate is preferably of unitary construction and in a generally symmetrical mask-like configuration contoured to permit it to conform to a patient's face when it is secured in position. It may be formed of plastic, rubber, metal, composite material, or other suitable materials having the desired physical properties for the application.

The securing apparatus 430 includes a generally cylindrically-shaped tower structure 435 extending in a substantially perpendicular direction from a top surface 438 of the plate 432 along axis A-A, the tower structure including a body portion 440 having a length and comprising a pair of oppositely disposed, pivotally interconnected, c-shaped collars 443, 446 respectively extending generally symmetrically about and along the axis in a direction away from the patient's face when installed on a patient, each of the collars having a length, first and second end portions 450, 450′ and 455,455′, an outer surface 457, 457′, and an inner surface 460, 460′. Each of the outer and inner surfaces extends intermediate the c-shaped collars' respective first and second end portions. Each of the collars has first and second end surfaces 462, 462′ and 464, 464′, a pair of generally parallel extending edge surfaces 465, 465′ and 466, 466′, the end surfaces, edge surfaces and the corresponding c-shaped collar each defining an opposed, semi-cylindrically shaped cavity 468, 468′ about the axis A-A. These cavities are most clearly shown in FIGS. 17-20.

Each c-shaped collar includes a plurality of substantially uniformly spaced-apart annular flanges 470 positioned axially along the respective inner surfaces thereof and extending substantially inwardly therefrom, and a plurality of structural recesses 474 positioned axially along each inner surface intermediate an adjacent two of the plurality of substantially uniformly spaced-apart annular flanges, each one of the plurality of annular flanges cooperating with an adjacent one of the plurality of annular flanges to define one of the plurality of structural recesses. Each of the annular flanges has an aperture 476 formed therein, each aperture being adapted to receive the airway device and a retention member when installed thereon, as will be described in greater detail below.

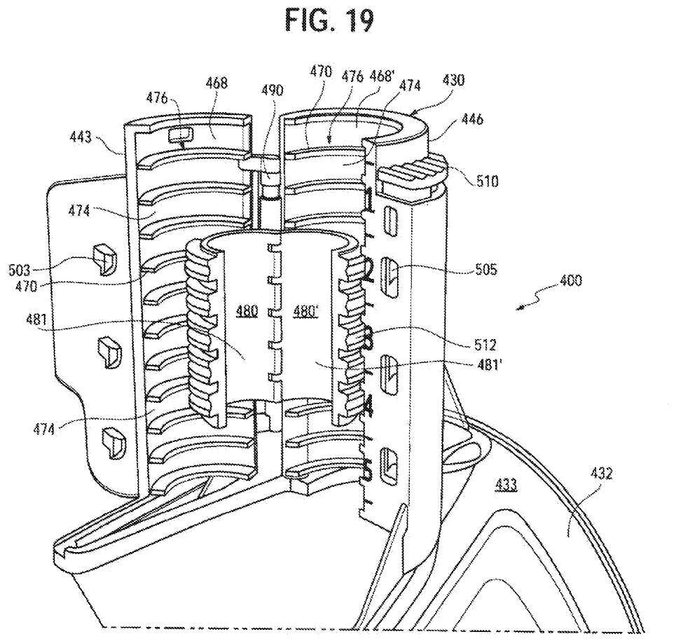

The interlocking, interactive catheter restraint system 400 of the instant embodiment further includes a pair of oppositely disposed, pivotally interconnected semi cylindrically or c-shaped retention members or collars 480, 480′ positioned in a semi-cylindrically shaped cavity 468, 468′ of collars 443, 446 respectively and extending circumferentially about and coaxially along the axis A-A. Each retention member or collar has a length, an inner surface 481, 481′ (FIG. 19) and an outer surface 482, 482′ and includes a plurality of substantially uniformly spaced-apart ribs 484 positioned axially along the length of the retention member and extending radially outwardly from the outer surface. Each of the inner surfaces may either be textured as hereinabove described with respect to the embodiment of FIGS. 11-14 and/or may be further coated with a suitable adhesive over which is placed a removable protective film 488, 488′. Each collar further includes a plurality of structural recesses 486 positioned axially along the length of the retention member, each of the plurality of structural recesses being positioned intermediate an adjacent two of the plurality of spaced-apart ribs.

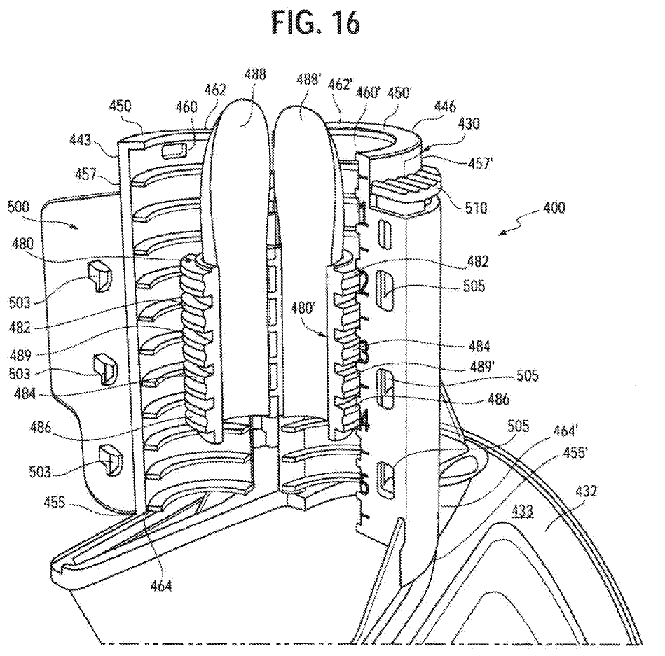

In operation, the c-shaped collars 443, 446 and the retention collars 480, 480′ are pivotally interconnected for example, by hinge member 490 and are moveable into mating contact with one another by closing the collars 443, 446 and thus rotatably urging the retention collars 480, 480′ into engagement with one another along a respective longitudinal edge 489, 489′ thereby forming a cavity 495; the cavity having an inner diameter and being adapted to engage the external surface 425 and continuous sidewall 420 of the airway device 410 such that axis of the airway device and the axis of the cylindrically shaped tower structure both extend coaxially along axis A-A. As shown in FIGS. 16-19, prior to placement of the airway device in the retention collars 480, 480′, the protective films 488, 488′ are pulled or peeled off of each of the inner surfaces 481, 481′, thus exposing the textured and/or adhesive-coated surface prior to rotating it into operative locking engagement with the external surface 425 and continuous sidewall 420 of the airway device 410, as illustrated in FIG. 21.

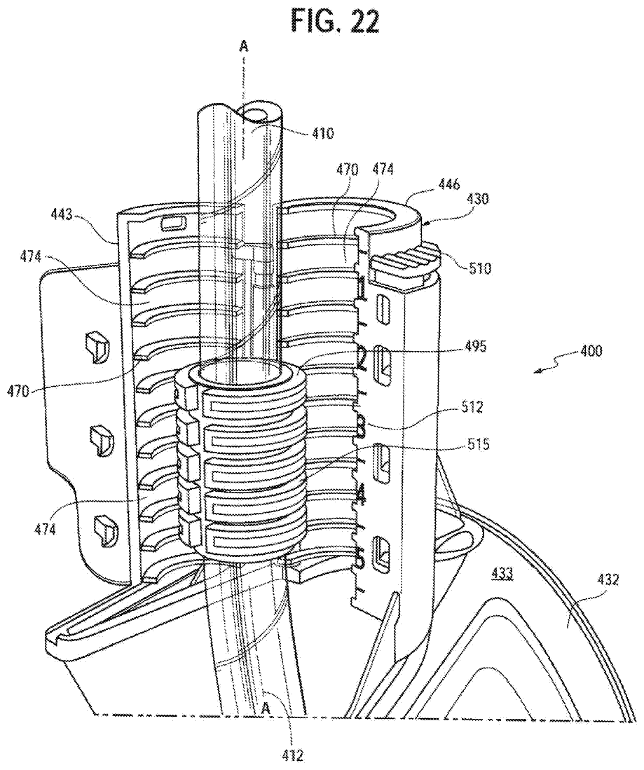

Each of the c-shaped collars 443, 446 includes a snap, clip, latch, camming operating apparatus or other suitable interlocking feature 500 having one or more locking members 503 adapted to releasably engage corresponding mating locking members 505 formed in or secured to the other c-shaped collar to releasably clamp them together circumferentially around the airway device in stabilizing and supporting engagement therewith. A release mechanism, for example, a quick-release actuator or button 510, allows the c-collars to be easily and rapidly released from locking engagement with one another to facilitate positioning, adjustment, and repositioning of the depth of insertion of the airway device into the patient's airway. A plurality of spaced apart reference markings or depth guides 512 are formed on c-collar 450′ and are structured and arranged to cooperate with other features of the system for ease of monitoring the relative position of the airway device with respect to the restraining tower, as defined more specifically below. For example, one or more of the plurality of ribs formed on the retention collars 480, 480′, by way of example, the middle rib 515, is marked to distinguish it from the other of the plurality of ribs formed thereon. Once the protective films have been removed, the encapsulated endotracheal tube is adhesively bonded to the retention collars 480, 480′ and, as shown in FIG. 22, the marked rib may be aligned by the attending practitioner with an appropriate one of the depth guides 512 to assist in inserting the airway device to a desired depth in the patient's trachea.

When the c-collars 450 and 450′ are locked together as shown in FIG. 15, the inwardly extending annular flanges 470 and structural recesses 474 on the inner surfaces of the of the c-shaped collars releasably engage corresponding mating structural recesses 486 and outwardly extending spaced-apart ribs 484 of the retention collars, thereby creating multiple points of contact and interaction between the securing apparatus and the airway device and thus preventing clinically significant movement of the airway device in response to substantial forces which may be applied thereto in any direction. The retention structure and securing apparatus cooperate to completely enclose the airway device whereby the airway device is isolated totally from any constricting, pinching, or crushing forces that would constrict an inner diameter thereof, thereby also restricting ventilation of a patient. Advantageously, in accordance with an embodiment of the instant invention, unlike prior art securing devices, the system herein disclosed may be secured laterally from a side of an airway device without being placed over the end of the device and, therefore, without disconnecting a ventilation source or interrupting ventilation of a patient. While the interactive restraint system of the embodiment of FIGS. 15-22 has been illustrated with respect to an endotracheal tube, it is to be understood that it may be used with equal efficacy to secure other types of catheters as herein described without departing from the scope of the instant invention.

While only selected embodiments have been chosen to illustrate the present invention, it will be apparent to those skilled in the art from this disclosure that various changes and modifications can be made herein without departing from the scope of the invention as defined in the appended claims. The interactive restraint system herein disclosed may be used in connection with any form of catheter in addition to cricothyrotomy, chest, and endotracheal tube configurations illustrated. Exemplary catheter devices also include tracheostomy tubes, Foley catheters, gastrostomy tubes, gastrojejunostomy tubes, nasogastric tubes, peripheral venous catheters, and peritoneal catheters, to name a few. Furthermore, the foregoing descriptions of the embodiments according to the present invention are provided for illustration only, and not for the purpose of limiting the invention as defined by the appended claims and its equivalents.

What is claimed is:

1. An interlocking restraint system for securing a catheter in a human patient or in an animal patient in veterinary applications, the patient having a body, a head, a face, a mouth, an oral cavity, a trachea, a neck, a cricothyroid membrane, a chest and a pleural cavity, the system comprising:
a catheter adapted to be maintained in a preselected position in a patient's body, the catheter including an elongate body having a first or patient end portion positioned in the patient's body, a second or machine end portion positioned outside the patient's body, an external or outer diameter and a continuous sidewall having an external surface extending intermediate the patient and machine end portions along and circumferentially about an axis;
a retention member or collar adapted to be adjustably positioned on the machine end of the catheter, the retention member including a body portion extending circumferentially about and coaxially along the elongate body of the catheter, the retention member body portion being adapted to be secured to the catheter body after positioning the catheter in the patient; and
a securing apparatus adapted to be secured to the patient, the securing apparatus being further adapted to releasably engage the retention member or collar and to cooperate therewith to maintain the catheter in position in the patient and to prevent movement thereof as a result of multidirectional forces being applied to the catheter.

2. The interlocking restraint system of claim 1 wherein the retention member includes an aperture extending therethrough, the aperture having an inner surface, an inner diameter, and circumference structured and arranged to fit over and in securing engagement with the continuous sidewall of the elongate body of the catheter.

3. The interlocking restraint system of claim 2 wherein the inner surface of the aperture includes surface treatments adapted to operatively connect the retention member to the continuous sidewall of the catheter.

4. The interlocking restraint system of claim 3 wherein the surface treatments comprise glues, pressure sensitive adhesives, surface texturing and scalloped surface patterns.

5. The interlocking restraint system of claim 1 wherein retention member has a length and includes at least one rib having oppositely disposed sides and extending circumferentially around the body portion of the collar and radially outwardly therefrom and a flat end portion operatively connected to and extending intermediate the sides in a direction generally parallel to the axis of the catheter, and a plurality of circumferentially extending structural recesses positioned axially along the length of the retention member, each of the structural recesses being positioned adjacent to a respective side of the at least one rib.

6. The interlocking restraint system of claim 1 wherein the retention member or collar comprises a tower structure including a body portion having a length and comprising a pair of oppositely disposed, pivotally interconnected, c-shaped collars respectively extending generally symmetrically about and along the axis of the catheter, each of the collars including first and second end portions, an outer surface, an inner surface, the outer and Inner surfaces extending intermediate the first and second end portions, a pair of generally parallel extending edge surfaces, the edge surfaces and the corresponding c-shaped collar each defining a semi-cylindrically shaped cavity extending about the axis.

7. The interlocking restraint system of claim 6 wherein each of the c-shaped collars includes at least one rib having oppositely disposed sides positioned axially along the respective outer surfaces thereof and extending circumferentially about and substantially outwardly therefrom and at least one structural recess positioned axially along each outer surface intermediate the spaced-apart annular flanges.

8. The interlocking restraint system of claim 7 wherein the inner surface of each c-shaped collar includes a layer of a bonding agent adapted to operatively connect the c-shaped collar to the catheter.

9. The interlocking restraint system of claim 8 wherein the bonding agent includes an adhesive or a double-sided tape.

10. The interlocking restraint system of claim 9 further including a protective film removably positioned over each layer of bonding agent, the protective film being adapted to be removed prior to positioning each of the c-shaped collars on the catheter.

11. The interlocking restraint system of claim 7 wherein the inner surface of each c-shaped collar includes a surface texturing.

12. The interlocking restraint system of claim 11 wherein the surface texturing is in the form of a scalloped surface pattern.

13. The interlocking restraint system of claim 1 wherein the securing apparatus includes a cylindrically shaped tower structure having a longitudinal axis, the tower structure including a body comprising a pair of pivotally interconnected or hinged semi-circular sections or c-shaped collars, each collar being operatively connected to a top surface of a base member and extending in a substantially perpendicular direction therefrom along and symmetrically about the longitudinal axis, each of the c-shaped collars having a length, first and second end portions, an outer surface, and an inner surface, each c-shaped collar defining an opposed, semi-cylindrically shaped cavity about the longitudinal axis.

14. The interlocking restraint system of claim 13 wherein each c-shaped collar of the securing apparatus includes a plurality of substantially uniformly spaced-apart annular flanges positioned axially along the respective inner surfaces thereof and extending substantially inwardly therefrom, and a plurality of structural recesses positioned axially along each inner surface intermediate an adjacent two of the plurality of substantially uniformly spaced-apart annular flanges, each one of the plurality of annular flanges cooperating with an adjacent one of the plurality of annular flanges to define one of the plurality of structural recesses.

15. The interlocking restraint system of claim 14 wherein each of the annular flanges has an aperture formed therein, each aperture being adapted to receive an elongate body of a catheter and a retention member.

16. The interlocking restraint system of claim 15 wherein the securing apparatus includes an adhesive pad adapted to be secured to a patient

17. The interlocking restraint system of claim 16 wherein the securing apparatus includes an interlocking device including a quick-release actuator.

18. The interlocking restraint system of claim 1 wherein the catheter comprises a cricothyrotomy tube.

19. The interlocking restraint system of claim 1 wherein the catheter comprises a chest tube.

20. The interlocking restraint system of claim 1 wherein the catheter comprises an endotracheal tube.
